(19) United States
(12) Patent Application Publication
(10) Pub. No.: US 2023/0081604 A1
(43) Pub. Date: Mar. 16, 2023

(54) TREATMENT OF ANASTOMOSES

(71) Applicant: PlantTec Medical GmbH, Luneburg (DE)

(72) Inventors: Joachim LAAS, Bad Bevensen (DE); Kai-Oliver MUTZ, Hannover (DE); Simon WEIGMANN, Lüneburg (DE)

(73) Assignee: PlantTec Medical GmbH, Luneburg (DE)

(21) Appl. No.: 17/965,157

(22) Filed: Oct. 13, 2022

Related U.S. Application Data
(63) Continuation-in-part of application No. 17/527,928, filed on Nov. 16, 2021.
Application No. 17/527,928 is a continuation of application No. 16/318,607, filed on Sep. 10, 2019, now abandoned.

(30) Foreign Application Priority Data
Jul. 18, 2016 (DE) 10 2016 008 533.9

(51) Int. Cl. A61K 31/718; A61P 17/02; A61K 9/00; A61K 9/06

(52) U.S. Cl. CPC A61K 31/718; A61P 17/02; A61K 9/0014; A61K 9/06

(57) ABSTRACT

Anastomotic healing, preferably after a surgical anastomosis, is improved by administering to a subject in need thereof a pharmaceutical composition comprising a carboxymethylated starch, a epichlorohydrin-modified starch, or a combination or mixture thereof. A disorder associated with surgical anastomosis, such as impaired bursting pressure, impaired wound healing, impaired growing together of anatomical structures such as intestinal sections, impaired coalescence effects in the joined anatomical structures such as intestinal sections, leakage of the intestine and infection of the intestine can be treated by administration of the aforesaid pharmaceutical composition.

### CROSS-REFERENCE TO RELATED APPLICATIONS

This application is a continuation-in-part of U.S. Ser. No. 17/527,928 filed on Nov. 16, 2021, which is a continuation of U.S. Ser. No. 16/318,607 filed on Sep. 10, 2019 (now abandoned), which is a National Stage Entry of PCT/DE2017/000201 filed on Jul. 7, 2017, which claims priority to DE10 2016 008 533.9 filed Jul. 18, 2016, each of which is incorporated herein by reference in entirety.

### FIELD OF THE INVENTION

This invention relates to a method for improving anastomotic healing. Furthermore, the invention provides a method of treating a disorder associated with surgical anastomosis, wherein said disorder can be impaired bursting pressure, impaired wound healing, impaired growing together of anatomical structures such as intestinal sections, impaired coalescence effects in the joined anatomical structures such as intestinal sections, leakage of the intestine and infection of the intestine.

### BACKGROUND ART

Anastomosis in general sense is a connection between two things (especially cavities or passages) that are normally diverging or branching, such as between blood vessels, leaf veins, or streams. Surgical anastomosis occurs when segments of intestine, blood vessel, or any other structure are connected together surgically (anastomosed). Examples include arterial anastomosis in bypass surgery, intestinal anastomosis after a piece of intestine has been resected, Roux-en-Y anastomosis and ureteroureterostomy. Conventional surgical anastomosis techniques include Linear Stapled Anastomosis, Hand Sewn Anastomosis and End-to-End Anastomosis. Anastomosis can be performed by hand or with an anastomosis assist device. Studies have been performed comparing various anastomosis approaches taking into account surgical time and cost, postoperative anastomotic bleeding, leakage, and stricture (Yao, Libin; Li, Chao; Zhu, Xiaocheng; Shao, Yong; Meng, Song; Shi, Linsen; Wang, Hui (2016-11-26). “An Effective New Intestinal Anastomosis Method”. Medical Science Monitor. 22: 4570-4576). In certain cases, pathological anastomosis may occur. Pathological anastomosis results from trauma or disease and may involve veins, arteries, or intestines. These are usually referred to as fistulas. In the cases of veins or arteries, traumatic fistulas usually occur between artery and vein. Traumatic intestinal fistulas usually occur between two loops of intestine (entero-enteric fistula) or intestine and skin (enterocutaneous fistula). Portacaval anastomosis, by contrast, is an anastomosis between a vein of the portal circulation and a vein of the systemic circulation, which allows blood to bypass the liver in patients with portal hypertension, often resulting in hemorrhoids, esophageal varices, or caput medusae.

Following resection of portions of the stomach or intestine and to maintain the respective bodily function, these sections are reconstructed by surgical anastomoses, usually by means of a mechanical connection between two anatomical structures, in particular by sewing them together. However, complications, such as impaired wound healing, impaired growing together without complications and impaired coalescence effects in the joined anatomical structures such as intestinal sections may occur. The risk of infection is also increased during surgical anastomosis. Another problem associated with surgical anastomosis is leakage of the intestine. The incidence of leakage from surgical anastomoses of the intestine is reported in the literature to be 0 to 20%, the mortality of this complication is reported to be 10 to 0 20%.

WO 2009/091549 A1 discloses in particular a hemostatic material, which is a modified starch having a molecular weight greater than 15,000 daltons. The modified starch is biocompatible, and is absorbed after degradation by amylases and carbohydrases. For the application, methods are disclosed in which the hemostatic material is applied to the skin 5 and internal tissue as a powder or a gel, for example, and is used for hemostasis, adhesion prevention, improved tissue healing, wound closure, and tissue bonding in open, endoscopic, laparoscopic, and laryngoscopic surgery and traumatology. The utility for treating anastomoses with this material is not disclosed.

Accordingly, there is a great need for expedients that support surgical anastomosis, and in particular to alleviate or prevent complications associated with surgical anastomosis.

### BRIEF DESCRIPTION OF THE INVENTION

In surgical anastomoses, it is desirable that rapid wound healing and growing together occur without complications. When topically applied to an anastomotic site, the pharmaceutical composition described herein provides excellent wound healing and coalescence effects in the joined anatomical structures such as intestinal sections, with exceptionally low risks of leakage and infection.

Unexpectedly, it has been shown according to the invention that a pharmaceutical composition comprising a certain carboxymethylated starch or a certain epichlorohydrin-modified starch or a combination or mixture thereof, is very well suited for improving anastomotic healing, accompanied by alleviating or preventing complications associated with surgical anastomosis, such as impaired bursting pressure, impaired wound healing, impaired growing together of anatomical structures such as intestinal sections, and impaired coalescence effects in the joined anatomical structures such as intestinal sections, and by reducing the risks of leakage and infection.

The invention therefore provides a method for improving anastomotic healing, preferably after a surgical anastomosis, comprising administering to a subject in need thereof an effective amount of a pharmaceutical composition comprising a carboxymethylated starch, epichlorohydrin-modified starch or a mixture thereof. Furthermore, the invention provides a method of treating a disorder associated with surgical anastomosis, wherein said disorder is selected from the group consisting of impaired bursting pressure, impaired wound healing, impaired growing together of anatomical structures such as intestinal sections, impaired coalescence effects in the joined anatomical structures such as intestinal sections, leakage of the intestine and infection of the intestine.

### BRIEF DESCRIPTION OF THE DRAWINGS

FIG. 1: Schematic representation of the gastric and/or intestinal wall.

FIG. 2a: Schematic representation of a surgical anastomosis.

FIG. 2b: Fold of anastomosis filled with polysaccharide gel.

FIG. 2c: Polysaccharide gel particles integrated in lamina propria of peritoneum supporting healing processes by activation of macrophages and fibroblasts.

FIG. 2d: Day 7 histology of a starch gel-treated anastomosis (PAS staining). Healed anastomosis is covered by an adhesion-free single-layer epithelium (1). There are no pathological findings on the mucosa (2). In the lamina propria of peritoneum few extracellular remnants of the starch gel (3) are seen, significant regenerating connective tissue with fibroblasts (4) has developed, also macrophages (5) and giant cells (6) are seen, in which starch gel is visible.

FIG. 3: Starch in powder form (left) and as a premixed gel (right).

FIG. 4: Microscopic image of the swollen powder particles/gel particles.

FIG. 5: Powder particles applied to intestine (left hand site), and as gel after sprinkling with an aqueous liquid (right hand site).

FIG. 6. Schematic sequence of a foreign body reaction

FIG. 7: Bursting pressure in mice seven (7) days after anastomosis and treatment of the anastomotic area with a carboxymethylated starch of the invention (treatment group) and saline (control group).

FIG. 8: Genexpression of the genes encoding Collagen-1 and Collagen-3 in mice seven (7) days after anastomosis and treatment of the anastomotic area with a carboxymethylated starch of the invention (treatment group) and saline (control group).

FIG. 9: Comparative water absorption test results.

FIG. 10: Water absorption test setup (On the left: scale on top of test cabinet with connective opening in the top through which the filter mount reaches to which in turn the filter is connected. The filter containing the test substance sits directly on the water surface. On the right: computer connected to the scale for data acquisition.)

### DETAILED DESCRIPTION OF THE INVENTION

### Definitions

Unless defined otherwise, all technical and scientific terms used herein have the same meaning as is commonly understood by one skilled in the art to which this invention belongs.

As used herein and in the claims, the singular form “a,” “an,” and “the” includes plural references unless the context clearly dictates otherwise.

The terms “treatment,” or “treating” or “ameliorating” as used in the specification and claims refer to an approach for achieving beneficial or desired results, including but not limited to a therapeutic benefit and/or a prophylactic benefit.

The term “therapeutic benefit” as used in the specification and the claims means eradication or amelioration of the underlying disorder being treated. A therapeutic benefit is achieved with the eradication or amelioration of one or more of the physiological symptoms associated with the underlying disorder such that an improvement is observed in the subject, notwithstanding that the subject may still be afflicted with the underlying disorder. For prophylactic benefit, the present compositions may be administered to a subject at risk of developing a particular affliction or disease, or to a subject reporting one or more of the physiological symptoms of a disease even though a diagnosis of the disease may not have been made.

The term “effective amount” refers to that amount of composition described herein that is sufficient to achieve the intended effect. The effective amount may vary depending upon the intended application or the subject and disease condition being treated, e.g., the weight and age of the subject, the severity of the disease condition, the manner of administration and the like, which can be determined readily by one skilled in the art. This term also applies to a dose that induces a particular response, e.g., reduction of adhesion. The specific dose may vary depending on the particular compounds that constitute the composition, the dosing regimen to be followed, timing of administration, and the physical delivery system in which the composition is carried.

Anatomical Physiological Basics for Anastomosis

In principle, the gastrointestinal tract is a continuous tube. Within the abdominal cavity from the diaphragm to the rectum, the wall of the tube consists of (from the inside to the outside):
mucous membrane,
muscle layer for transporting the bowel contents in the form of peristaltic waves
peritoneum, the outer layer of the tube.

The peritoneum is a three-layered skin consisting of:
Serosa epithelium or mesothelium (lamina epithelialis serosae). This is a single-layered squamous epithelium, a loose association of cells with many pores that allow exchange of fluids and substrates.
Lamina propria serosae. This is a layer with connective tissue fibers, fibrocytes, blood vessels, lymphatic vessels, nerve fibers, cells of the immune system (macrophages, lymphocytes, plasma cells).

Basics of Techniques for Intestinal Anastomoses.

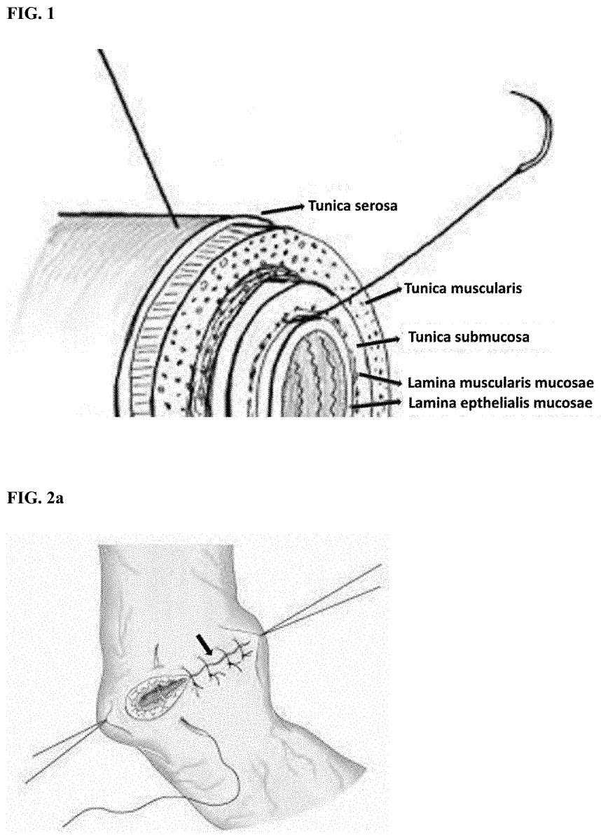

Anastomoses to the stomach and intestine are used to connect lumina of the stomach and/or intestine. The anastomoses must be designed in such a way that gastric and intestinal contents can pass without obstruction. It must also be ensured that no bowel contents can leak. The middle and outer layers of the intestinal wall are connected with sutures or staples in such a way that a mechanical connection is created that resists the internal pressure of the intestine. For sealing to the outside, the adaptation of the peritoneum is essential. As shown in FIG. 1, the muscular layer and peritoneum of the gastric and/or intestinal wall are bound with a suture.

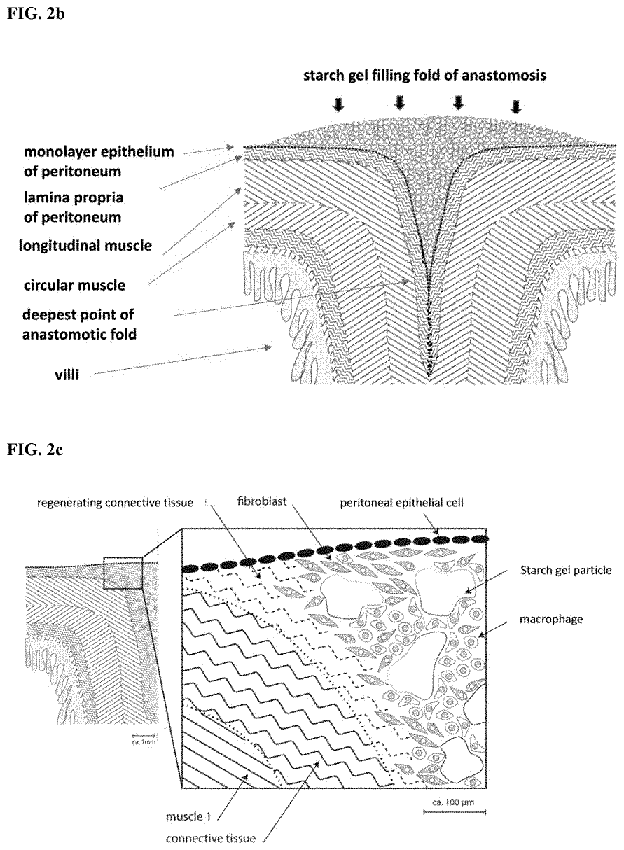

By tying the seam, the middle and outer walls are adapted. The ends of the tube are always turned inward. This results in an inward fold in all cases (see arrow in FIG. 2a). This also occurs with staple suturing instruments. In the zones where the suture or staples adapt the intestinal walls, blood flow is reduced by the pressure of the sutures or staples.

FIG. 2b shows schematically the wall layers of intestines after completion of an anastomosis. The inward fold is filled with starch gel particles.

FIG. 2c schematically shows the healing process within the first 2 days after application of starch gel on intestinal anastomoses. The starch particles are surrounded by macrophages and fibroblasts. The healing zone in the former anastomotic fold is already covered by a layer of peritoneal epithelial cells.

FIG. 2d is the histology of an intestinal anastomosis healed with starch gel support. Histological examinations at seven days show excellent healing with abundant fresh connective tissue and fibroblasts in the lamina propria of the peritoneum. There are also remnants of the starch gel both extracellularly and intracellularly in macrophages and giant cells. The healing zone is covered with an intact single layer peritoneal epithelium. The histological findings fit well with the tests showing better bursting pressure and significantly increased gene expression for Col1 and Col3 after anastomosis compared to animals without treatment.

The Pharmaceutical Composition

A pharmaceutical composition useful for practicing the method of the invention comprises carboxymethylated starch or epichlorohydrin-modified starch, or a combination or mixture thereof. Surprisingly, it has been found that the administration of such a type of starch to a subject provides excellent results in improving anastomotic healing. This applies in particular for anastomoses following a surgery in and/or on the intestine, but also for surgical anastomoses of mesenchymal organs, for example and in particular bones, tendons, muscles, fat tissue, and connective tissue, wherein connective tissue is understood to mean tissue that is present between two different tissues and/or organs and joins them together; for example, tendons are the connective tissue between bones and muscles.

The pharmaceutical composition can be advantageously present in the form of a gel; in addition, the pharmaceutical composition advantageously can include water and/or at least one salt, for example and in particular an alkali metal salt such as sodium chloride or potassium chloride.

In a preferred embodiment, the pharmaceutical composition comprises carboxymethylated starch, and optionally water and/or at least one alkali metal salt, e.g., sodium chloride or potassium chloride.

In another preferred embodiment, the pharmaceutical composition includes epichlorohydrin-modified starch, and optionally water and/or at least one alkali metal salt, e.g., sodium chloride or potassium chloride.

In a further preferred embodiment, the pharmaceutical composition comprises a combination or mixture of carboxymethylated starch and epichlorohydrin-modified starch, and optionally water and/or at least one alkali metal salt, which can be sodium chloride or potassium chloride.

In practice, it has proven beneficial for the carboxymethylated starch or the epichlorohydrin-modified starch to have a molecular weight of 500,000 daltons to 11,000,000 daltons, and/or for the starch particles, the carboxymethylated starch or the epichlorohydrin-modified starch particles to have a particle diameter in the range from 30 μm to 100 μm.

Accordingly, the invention provides a method of treatment utilizing pharmaceutical composition comprising carboxymethylated starch or epichlorohydrin-modified starch or a mixture or combination thereof, wherein said carboxymethylated starch or epichlorohydrin-modified starch has a molecular weight in the range from 500,000 daltons to 11,000,000 daltons, and/or wherein the starch particles of the carboxymethylated starch and/or the epichlorohydrin-modified starch particles have a particle diameter in the range of 30 microns to 100 microns.

Preferably, said carboxymethylated starch or epichlorohydrin-modified starch has a molecular weight in the range from 750,000 daltons to 10,000,000 daltons or 1,000,000 daltons to 9,000,000 daltons.

More preferably, said carboxymethylated starch or epichlorohydrin-modified starch has a molecular weight in the range from 2,000,000 daltons to 8,000,000 daltons or 3,000,000 daltons to 7,000,000 daltons.

Most preferably, said carboxymethylated starch or epichlorohydrin-modified starch has a molecular weight in the range from 4,000,000 daltons to 6,000,000 daltons.

Results in the improvement of anastomotic healing have been obtained with a pharmaceutical composition that comprises carboxymethylated starch and optionally water and/or at least one salt, selected from the group consisting of sodium chloride or potassium chloride, wherein said carboxymethylated starch has a molecular weight in the range from 500,000 daltons to 11,000,000 daltons, and/or wherein the starch particles of the carboxymethylated starch have a particle diameter of 30 microns to 100 microns.

The carboxymethylated starch of the pharmaceutical composition has the following, further preferred characteristics: It is a crosslinked, carboxymethylated starch containing approximately 20 wt % amylose and approximately 80 wt % amylopectin in a relatively constant ratio. The amylose is rather amorphous, and the amylopectin represents the crystalline portion of the starch. The modified starch is present as a salt-starch glycolate, and is generally the sodium salt of a carboxymethyl ether of the starch.

A starch particle of the carboxymethylated starch is made up of about 4.5×10¹⁰ to 2.3×10¹² amylose molecules and 5.6×10⁷ to 1.3×10¹⁰ amylopectin molecules. The degree of crosslinking of the carboxymethylated is typically in the range from 25 to 45%.

The carboxymethylated starch may contain the at least one salt, which is selected from the group consisting of sodium chloride and potassium chloride, preferably sodium chloride, in an amount up to 10 weight %, preferably between 2 weight % and 5 weight %, calculated on the basis of the dry mass of the carboxymethylated starch.

After addition of water, or when the pharmaceutical composition is in gel form, the pH value of the solution or gel is in the range from 3.0 and 7.5, preferably from 5.0 to 7.5, most preferably from 6.5 to 7.5.

The carboxymethylated starch is present as a white or almost white fine, free-flowing powder. The powder is very hygroscopic, and is practically insoluble in methylene chloride. It forms a translucent suspension in water.

The powder is made up of irregularly shaped, oval or pear-shaped particles in a size range from 30 to 100 or 10 to 35 μm with rounding.

The starch particles have typically a surface of approximately 0.2 m²/g. Occasionally occurring clusters of particles are made up of two to four particles. The particles have an eccentric hilus and clearly visible concentric grooves. The particles exhibit a distinct black cross on the hilus, between intersecting Nicol prisms. Small crystals are discernible at the surface of the particles. The particles exhibit significant swelling, up to approximately 30 times their own weight, upon contact with water or salt solutions.

The carboxymethylated starch comprised in the pharmaceutical composition can be produced using the following method:

The synthesis of the carboxymethylated starch generally takes place in three steps, in each case as a suspension, wherein Step 1 comprises crosslinking of the starch, Step 2 comprises carboxymethylation of the starch, and Step 3 is a neutralization step.

The crosslinking-step uses hydroxyl groups attached to starch (present in the basic glucose building blocks of the starch). The reaction of these hydroxyl groups with multifunctional reagents results in crosslinked starches. In a granule of starch, many chains are found in closer proximity, and such reactions not only take place between single chains, but it links side by side chains as well. These crosslinking agents form either ether or ester inter-molecular linkages between hydroxyl groups on starch molecules A small amount of multifunctional reagent is enough to interconnect starch molecules by crosslinking reactions. Methods for crosslinking of starches are generally known to the person skilled in the art. An overview of starch crosslinking is e.g. given in Shah, Nimish & Mewada, Rajubhai & Mehta, Tejal. (2016). Crosslinking of starch and its effect on viscosity behaviour. Reviews in Chemical Engineering. 32. 10.1515/revce-2015-0047, which is incorporated herein in entirety.

Since the reactions take place in suspension, the substitution is inhomogeneous. Outer areas of the starch are more greatly affected by the modifications than the core areas of the starch structures. The reactions are influenced by diffusion within the starch structure and accessibility to the starch structure. Amorphous areas are more easily accessible than crystalline areas, which are less strongly modified. From a chemical standpoint, both reactions are nonspecific, and, provided that diffusion inhibition or steric hindrance is present, have no preferred reaction pattern for the individual hydroxyl groups of a glucose unit. Theoretically, the degree of substitution is 3 (with substitution of all three OH groups of a glucose unit as the monomeric building block of the starch). The actual degree of substitution appears to be in the range of about 0.2 to 0.4; i.e., one OH group is substituted in approximately every fourth glucose unit (not taking crosslinking into account). This degree of substitution results from the values for the bound salt.

After Steps 1 and 2 have been performed, neutralization is carried out as Step 3, using an acid. Preferably, said acid is selected from the group consisting of succinic acid, phosphoric acid, hydrochloric acid, sulfuric acid, and citric acid. The selection of the acid has an effect on inorganic and organic substances that can be detected as residues in the modified starch. Most preferred according to the invention is phosphoric acid.

Accordingly, the pharmaceutical composition may further comprise an organic or inorganic salt resulting from the neutralization step, wherein said organic or inorganic salt is selected from the group consisting of succinate, phosphate, chloride, sulphate, and citrate.

Mode of Administration to a Subject

In the simplest form of the method according to the invention the pharmaceutical composition is administered in powder form directly after an anastomosis and directly on the anastomosis area or site to form a gel covering the anastomosis area or site, or is mixed with an aqueous liquid and optionally salts to form a gel, which is then administered in gel form on the anastomosis area or site. When administered in powder form directly on the anastomosis area or site, a gel is formed in situ due to the presence of residual body fluids at the anastomosis area or site, or by drizzling the powder after administration on the anastomosis area or site with an aqueous liquid.

More specifically, the methods of the invention comprise the following steps:
i) providing a powder of starch particles of carboxymethylated starch or epichlorohydrin-modified starch or a combination or mixture thereof;
ii) administering the powder of Step 1 directly on a anastomosis area or site in a subject; and
iii) forming a gel covering the anastomosis area or site
   by residual body fluids present at the anastomosis area or site; or
   by drizzling the powder after administration on the anastomosis area or site with an aqueous liquid.

FIG. 5 shows starch powder particles applied to the intestine (left hand site) and as a gel after sprinkling with an aqueous liquid (right hand site).

In an alternative embodiment, the methods of the invention comprise the following steps:
i) providing a powder of starch particles of carboxymethylated starch or epichlorohydrin-modified starch or a combination or mixture thereof;
ii) premixing the powder of Step i) with an aqueous liquid;
iii) forming a gel; and
iv) administering the gel of Step iii) on the anastomosis area or site in a subject.

In a further alternative embodiment, the method of the invention comprises the following steps:
i) providing a pharmaceutical composition which comprises a gel of carboxymethylated starch or epichlorohydrin-modified starch or a combination or mixture thereof; and
ii) administering the gel of Step i) on the anastomosis area or site in a subject.

When the pharmaceutical composition used in the above described methods of the invention comprises a combination of carboxymethylated starch and epichlorohydrin-modified starch, the weight ratio of carboxymethylated starch to epichlorohydrin-modified starch is preferably in the range of 99:1, more preferably in the range of 90:10, 80:20, 70:30 or 60:40, most preferably 50:50.

In an alternative embodiment, when the pharmaceutical composition used in the above described methods of the invention comprises a combination of carboxymethylated starch and epichlorohydrin-modified starch, the weight ratio of epichlorohydrin-modified starch to carboxymethylated starch is preferably in the range of 99:1, more preferably in the range of 90:10, 80:20, 70:30 or 60:40, most preferably 50:50.

The aqueous liquid is preferably selected from the group consisting of water, and saline solution, such as isotonic saline solution, or hypotonic, isotonic, or hypertonic saline solution, as the liquid phase. The aqueous liquid can contain one or more cations selected from the group consisting of sodium, potassium, ammonium, magnesium, calcium, iron(II), iron(III), aluminum; and/or one or more anions selected from the group consisting of fluoride, chloride, bromide, iodide, oxide, sulfide, carbonate, sulfate, phosphate, nitrate, chromate, permanganate, hexacyanoferrate(II). Ringer's solution, Ringer's acetate solution, and Ringer's lactate solution may also be used as the liquid phase.

Accordingly, the pharmaceutical composition, when in gel form, may further include one or more cations selected from the group consisting of sodium, potassium, ammonium, magnesium, calcium, iron(II), iron(III), and aluminum; and/or further including one or more anions selected from the group consisting of fluoride, chloride, bromide, iodide, oxide, sulfide, carbonate, sulfate, phosphate, nitrate, chromate, permanganate, hexacyanoferrate(II), acetate and lactate.

Most preferably, the aqueous liquid is water or isotonic saline.

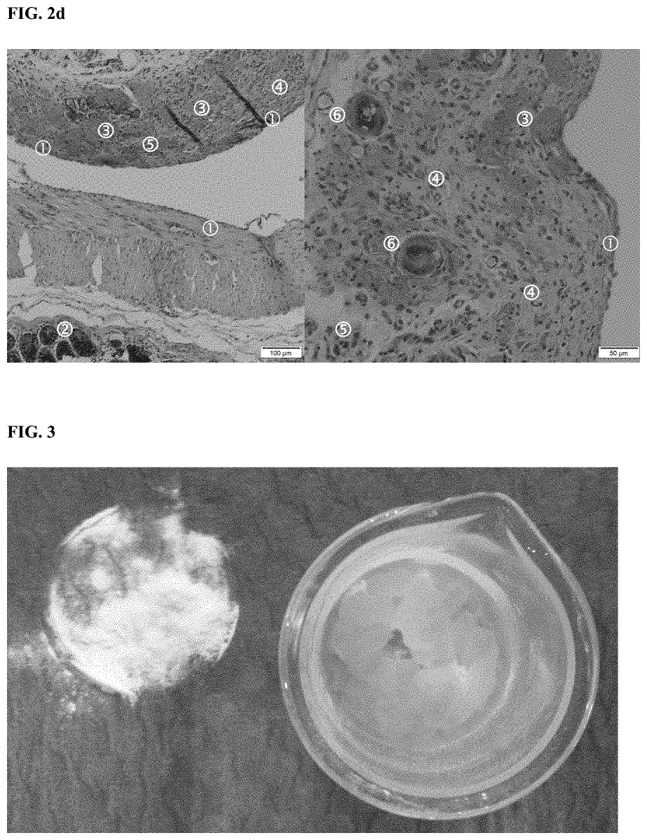

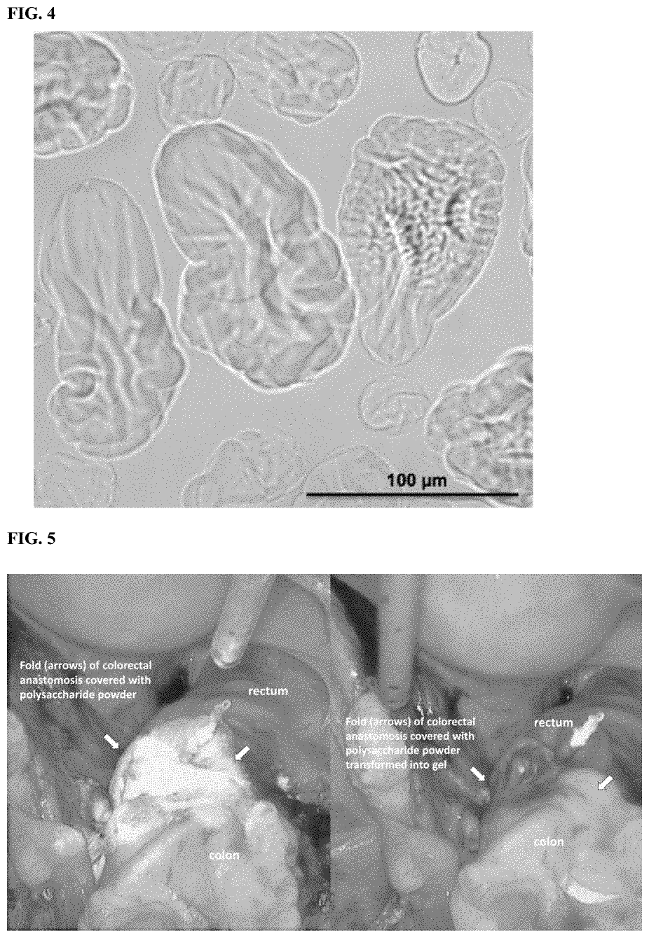

In the methods of the invention as described hereinbefore, the aqueous liquid, most preferably water or isotonic saline, is suitably added in a volume of 1-40 ml per gram powder of the pharmaceutical composition. An amount of aqueous liquid is added to the starch powder, sufficient to form a translucent gel. FIG. 3 shows a starch of the invention in powder form (left) and as a premixed gel (right). FIG. 4 shows a microscopic image of the swollen powder particles/gel particles.

The pharmaceutical composition of the invention is applicable in veterinary and human medicine. The subject is preferably a mammal. Most preferably, the subject is a human and the pharmaceutical composition of the invention is applied in human medicine.

Mode of Action of the Pharmaceutical Composition

It is presumed that the excellent properties are based on the following mechanisms:

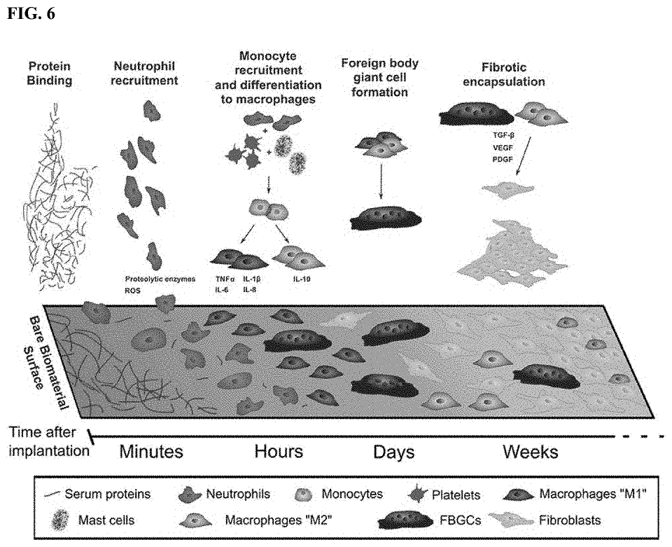

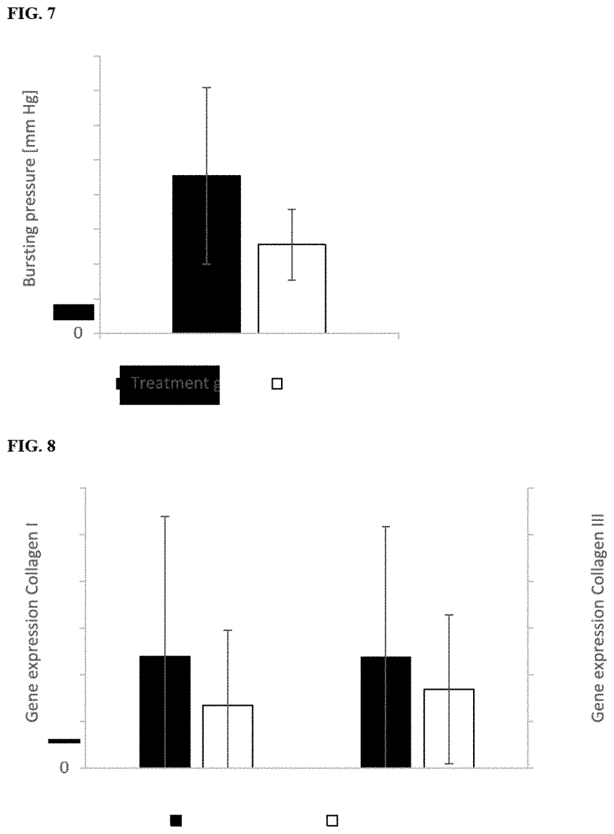

As a result of the administration carboxymethylated starch or epichlorohydrin-modified starch or a combination or mixture thereof on an anastomotic site of a subject according to the invention, an early body reaction is triggered, which is accompanied by the migration of macrophages, followed by fibroblasts, which as stabilizing factors are responsible for improved anastomotic healing with improved performance. The cascade of events is shown in FIG. 6.

Within minutes, endogenous proteins bind to the foreign swollen starch particles in the starch gel, followed by neutrophilic leukocytes. Within hours, monocytes are recruited and differentiate into macrophages. After only one day, the first fibroblasts are present. In the case of nonabsorbable foreign bodies, the process leads to fibrotic encapsulation of the foreign body(s).

The starch particles applied to the anastomoses trigger the cascade of foreign body reaction including mobilization of fibroblasts. However, the starch particles are reabsorbed within a week, so the components of the foreign body reaction, including fibroblasts, can be used for the healing process of the intestinal anastomosis. Since the foreign bodies have disappeared after one week, the later steps of the foreign body reaction, especially the processes of fibrous encapsulation, no longer take place.

Specifically, the gel particles, which rest on the intestinal surface, enter the lamina propria through the pores of the epithelium. Here, the many particles trigger a multilocular early foreign body reaction, which has a general course triggered by interleukins.

As the result of administering the medicament according to the invention, i.e., the carboxymethylated starch or the epichlorohydrin-modified starch or mixture or combination thereof, a gel is acting locally on the anastomotic area or site. The presence of the swollen starch powder particles in starch gel is associated with an increased invasion of cellular components of early healing. Activated macrophages come into contact with the surfaces of the swollen starch particles quite rapidly, forming a framework, followed by other cellular components of early healing. Although the starch framework/matrix is degraded within days, an increased number of cellular components remain in the healing area, resulting in improved wound healing, wherein in particular an increased number of macrophages for the macrophagocytosis of bacteria, apoptopic cells, necrotic cells, cells with foreign protein, and polysaccharide surfaces are observed in the anastomotic area. In addition, an increased number of fibroblasts and other cellular components of wound healing are also observed. The presence of cells for early healing and the subsequent wound healing processes are accelerated by these factors. These cellular components form the basis for improving anastomotic healing, and also provide better resistance to increased intraluminal pressure or other mechanical stresses in the intestinal area, such as resulting in improved bursting pressure resistance and reduced occurrence of leaks in intestinal anastomoses.

Histological analyses in an area have shown improved submesothelial healing after one week due to application of the above-mentioned starch gel, i.e., in a form of the medicament according to the invention showing remnants (residues) of hydrobeads, macrophages and giant cells, fibroblasts, and emerging connective tissue.

The improvement in healing results from the presence of the pharmaceutical composition according to the invention, which activates the early cellular healing processes, i.e., the attraction of macrophages, followed by the attraction of fibroblasts. The latter represent stabilizing factors for improving anastomotic healing with improved performance, i.e., better resiliency against mechanical stress and reduced occurrence of anastomotic insufficiency.

Surprisingly, it has been found that the administration of the pharmaceutical composition according to the invention leads to an improved bursting pressure of the colon or intestine, accompanied by a significantly increased gene expression for Type I collagen (COL1) and Type 3 collagen (COL3) one week after the anastomoses were created. Moreover, the hydroxproline content at the anastomosis site is increased.

Type I collagen is the most abundant collagen of the human body. It forms large, eosinophilic fibers known as collagen fibers. It is present in scar tissue, the end product when tissue heals by repair, as well as tendons, ligaments, the endomysium of myofibrils, the organic part of bone, the dermis, the dentin, and organ capsules. In humans, Type 1 collagen is encoded by the COL1A1 and COL1A2 genes.

Type III collagen is a homotrimer, or a protein composed of three identical peptide chains (monomers), each called an alpha 1 chain of type III collagen. Formally, the monomers are called collagen type III, alpha-1 chain and in humans are encoded by the COL3A1 gene. Type III collagen is one of the fibrillar collagens whose proteins have a long, inflexible, triple-helical domain.

Hydroxyproline is an amino acid, which is obtained by the hydrolysation of proline. It plays a role in the synthesis of collagens. Collagens contain hydroxyproline to a certain extend as building block in the polypeptide chain.

The increased content of COL1, COL3 and hydroxyproline in the tissue at the anastomotic area is therefore an indicator of the improved anastomosis healing effect of the pharmaceutical composition of the invention.

Therefore, the methods of the invention further contain the step of assessing anastomotic wound healing by functional analyses determining the strength of the anastomosis from anastomotic bursting pressures and/or histologically, and by collagen production shown by gene expression of Collagen-1 (Col1) and Collagen-3 (Col3) and hydroxyproline content.

The pharmaceutical composition according to the invention is further advantageous compared to conventional products, because it shows a reduced side effects as the potential to trigger necrosis is reduced. Necrosis is a form of cell injury which results in the premature death of cells in living tissue by autolysis. Necrosis is caused by factors external to the cell or tissue, such as infection, or trauma which result in the unregulated digestion of cell components. In contrast, apoptosis is a naturally occurring programmed and targeted cause of cellular death. While apoptosis often provides beneficial effects to the organism, necrosis is almost always detrimental and can be fatal.

Necrosis is a known side effect of hemostatic products, such as Starsil®, Perclot® or Haemocer® due to their high capacity of water absorption. These products trigger necrosis through local dehydration of intestinal surfaces. The effect on causing necrosis is undoubtedly greater the more water a product can adsorb.

As shown in Example 2, the water absorption of the pharmaceutical composition according to the invention is much lower than of other commercially available products. Preferably, the pharmaceutical composition according to the invention shows a water absorption ability in the range of 10 fold to 22 fold (1,000 to 2,200%) of its dry weight after 10 min exposure to an aqueous solution. More preferably, the pharmaceutical composition according to the invention shows a water absorption which is lower than 21 fold (2,100%) after 10 min exposure to an aqueous solution. Most preferably, the pharmaceutical composition according to the invention shows a water absorption which is lower than 20 fold (2,000%), lower than 19 fold (1,900%), lower than 18 fold (1,800%), lower than 17 fold (1,700%), lower than 16 fold (1,600%) or lower than 15 fold (1,500%) after 10 min exposure to an aqueous solution.

Moreover, it was surprisingly discovered throughout the invention that the danger of generating necrosis can be further reduced, when the pharmaceutical composition of the invention is not applied in powder form on the anastomosis area or site, but as a gel after mixing with an aqueous solution, preferably water. Therefore, in a preferred embodiment, the carboxymethylated starch of the present invention is preferably administered on the anastomosis area or site as a gel.

The subject matter according to the invention is explained in the following embodiment by way of two examples and in a nonlimiting manner.

### Example 1: Use of a Carboxymethylated Starch to Support of Colonic Anastomotic Healing in Mice

Naïve mice underwent anastomosis surgery or laparotomy with a subsequent treatment with carboxymethylated starch (treatment group) or saline (NaCl, control group).

The carboxymethylated starch used had the following characteristics:
Chemical name: Starch, carboxymethyl ether, sodium salt, present as sodium glycolate
Appearance: white or almost white fine, free-flowing powder
Odour: neutral
Molecular weight: in the range of 500,000 daltons to 11,000,000 daltons
Particle size: in the range of 30 μm to 100 μm
Water content: 3.9%
Sodium chloride: 4.0%
Sodium glycolate: <2.0%
pH (as 3% solution): 6.7

The carboxymethylated starch was applied to the anastomosis area in the mice of the treatment group as powder.

The NaCl was applied to the anastomosis area in the mice of the treatment group as aqueous solution containing 0.9 weight % NaCl.

Seven days after surgery, tissue was isolated for further analysis including functional analysis of anastomosis bursting pressure, and gene expression of Collagen-1 and Collagen-3 and biochemical hydroxyproline measurement. The results are summarized in Table 1.

TABLE 1
Results of the treatment of anastomosis areas
with carboxymethylated starch and saline

| | Treatment | |
|---|---|---|
| Analysis | group | Control group |
| BP* | 227 ± 127 | 128 ± 51 |
| Col1 GE** | 11.96 ± 15 | 6.76□8 |
| Col3 GE*** | 5.95□7 | 4.22□4 |
| HP content**** | 120.9□23.9 | 109.1□32.0 |

*Bursting pressure (mm Hg)
**Collagen-1, rel. expression [mRNA]
***Collagen-3 rel. expression [mRNA]
****Hydroxyproline content

Anastomotic bursting pressure (ABP) was measured on postoperative Day 7. The abdominal incision was reopened and colonic anastomosis was identified and gently dissected away from the surrounding tissues. A 3 cm segment including the cecum and ascending colon with the anastomotic site was dissected with the sutures left in place to preserve the integrity of the anastomosis. The anastomotic specimen was cleared from feces and ligated twice at the cecal site using a Silk 5-0 (Braun, Melsungen, Germany) suture. An 18 G arterial catheter (Vygon, France) was inserted intraluminal within the ascending colon at the distal end, and two sutures were tied to prevent leakage. The other end of the catheter was connected in an in vitro organ bath setup (Heap Labor Consult, Bovenden, Germany) to an infusion pump (Braun, Melsungen, Germany), and isotonic saline solution was infused at a constant rate of 200 ml/h. The intraluminal pressure (millimeters of mercury) was measured and recorded using a pressure transducer with an amplifier and analyzed using the Biopac A/D systems (AcqKnowledge® Software, Biopac Systems, Santa Barbara, Calif., USA). ABP was indicated as a sudden loss of pressure and defined as the maximum intraluminal pressure prior to leakage.

Comparing the anastomotic bursting pressure of the animals in the treatment group versus animals in the control group, there was a 77.3% increase of bursting pressure indicating an acceleration of healing in the presence of the carboxymethylated starch of the invention (FIG. 5).

Anastomotic wound healing can be assessed by functional analyses determining the strength of the anastomosis from anastomotic bursting pressures, histologically by using an anastomosis wound healing score, and by collagen production shown by gene expression of Collagen-1 (Coll) and Collagen-3 (Col3) and hydroxyproline content (Pantelis D, Kabba M S, Kirfel J, et al (2010) Transient perioperative pharmacologic inhibition of muscularis macrophages as a target for prophylaxis of postoperative ileus does not affect anastomotic healing in mice. Surgery 148:59-70).

Comparing gene expression of Collagen-1 and Collagen-3 in animals with and without treatment with carboxymethylated starch there was a 76.9% increase for Collagen-1 and a 41% increase for Collagen-3 expression. There was also a slight increase of 10.8% for hydroxyproline content. Especially the marked increase of Collagen-1 and Collagen-3 gene expression is related to the use of the carboxymethylated starch and strongly supports the anastomotic healing processes.

Conclusion: The carboxymethylated starch supports the anastomotic healing process in mammals.

### Example 2: Comparative Water Absorption Tests

Water absorption ability has been tested for the following starch products:
i. Carboxymethylated starch as used in Example 1;
ii. StarSil®
iii. Perclot®
iv. Haemocer®

Starsil® Hemostat is a plant-based medical product composed of fine powder particles, which are ultra-hydrophilic and absorb water from the blood. This dehydration results in a high concentration of blood components such as thrombocytes, red blood cells and coagulation proteins and thus accelerates the natural blood clotting process. It is indicated in surgery as an adjunctive hemostat to control capillary, arterial or venous bleeding in situations where the use of ligatures, pressure or other conventional methods prove to be inadequate or impractical. It is manufactured by Hemotec Medical GmbH (Germany).

Perclot® is a Polysaccharide Hemostatic System (PHS), which is indicated for use in surgical procedures (except neurological and ophthalmic) or injuries as an adjunct hemostat when control of bleeding from capillary, venous, or arteriolar vessels by pressure, ligature, and other conventional means is either ineffective or impractical. It is provided by Baxter International Inc.

Haemocer® is an Absorbable Polysaccharide Haemostat (APH). Upon contact with blood HaemoCer™ enhances the natural clotting cascade by rapidly dehydrating the blood and accelerating the concentration of platelets, red blood cells and coagulation proteins at the bleeding site. It is manufactured by BioCer Entwicklungs-GmbH (Germany).

Experimental Conditions

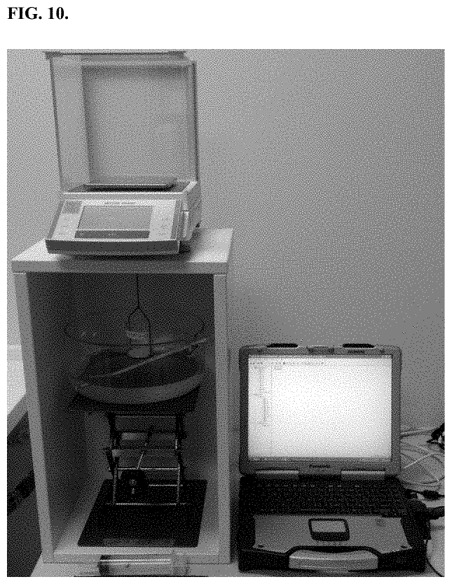

The water absorption testing is performed using the setup depicted in FIG. 10.

The setup is as follows:
1. The test cabinet is placed on a laboratory table, with the central opening on one front side turned upward. The scale is put above this opening. The lab jack is put on the lower rack. The glass tank is placed on the platform of the lab jack and filled with 2 liters of lab water cat. 3.
2. The calibration status of the scale (Mettler Toledo XS100355) is checked
3. The correct amount of test substance is weighed out and put in a safe place.
4. The filter mount (length: diameter 50.5 mm) is attached to the hook on the lower side of the scale (from below through the opening of the test cabinet). The lab jack is cranked up until about 1 cm below the end of the filter mount. A filter (glass filter crucibles Duran 50 ml, porosity category 3, order No. 258513303) is placed on the mount. The scale is started and tared. The laboratory computer is started, connected to the scale, and the data retrieval software (Mettler Toledo BalanceLink) is started.

Subsequently the filter is tested in the following manner:

The syringe is filled with more than 50 ml of lab water cat. 2. The scale is tared. The data retrieval software is started on the computer. The data recording is started. More than 50 ml of water are poured into the filter and recording proceeds until the scale shows a weight of less than 25.0 g. The time it takes for 25 ml to pass the filter is calculated. If it takes longer than 10 minutes, the filter is discarded. Otherwise, the tested filter can be sued. The filter is emptied and dabbed dry with a lint-free cloth. The filter plate can be kept soaked with water (as it will absorb water anyway).

The water absorption capacity measurement is then performed as follows:
1. The tested filter is placed in the filter mount. The scale is tared.
2. The tested filter is lowered until it touches the water surface. The scale will show negative weight, as the buoyancy changes the weight of the filter setup. The filter is lowered further, until the scale shows 0.0 g again.
3. Data recording is started.
4. With a spatula of the appropriate size, the required amount of substance is poured into the filter, all at once. Data is recorded for at least 15 minutes or as long as required.
5. After the end of the recording time, the filter is raised out of the water and the final weight is recorded. The logging of the recording is stopped.

Results

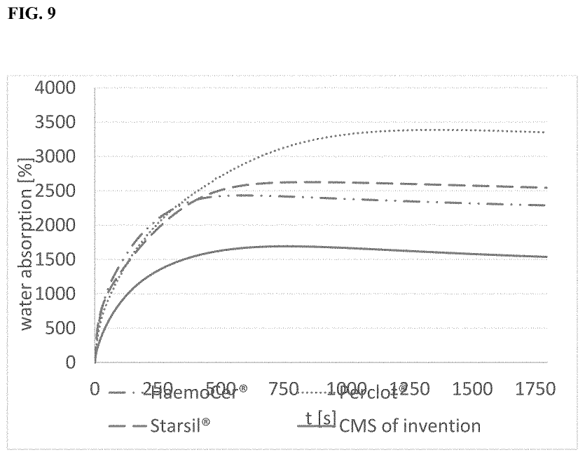

As can be seen in FIG. 9, the water absorption ability of the tested starch products is as follows:

| | Water absorbency, % (fold) | | |
|---|---|---|---|
| Product | After 10 min. | After 20 min. | After 30 min. |
| CMS | 1,671 (16.7) | 1,632 (16.3) | 1,538 (15.4) |
| Starsil ® | 2,584 (25.8) | 2,605 (26.1) | 2,545 (25.5) |
| Perclot ® | 2,905 (29.1) | 3,377 (33.8) | 3,351 (33.5) |
| Haemocer ® | 2,433 (24.3) | 2,354 (23.5) | 2,288 (22.9) |

CMS . . . carboxymethylated starch

1. A method for promoting tissue healing after anastomosis comprising administering to an anastomotic site of a subject an effective amount of a pharmaceutical composition which is a member of the group consisting of carboxymethylated starch, epichlorohydrin-modified starch, and a mixture thereof.

2. The method of claim 1, wherein said anastomosis is surgical anastomosis.

3. The method of claim 2, wherein said method comprises the treatment of a disorder associated with surgical anastomosis, selected from the group consisting of impaired bursting pressure, impaired wound healing, impaired growing together of anatomical structures, impaired coalescence effects in joined anatomical structures such as intestinal sections, leakage of intestine, and infection of intestine.

4. The method of claim 1, wherein said pharmaceutical composition is administered as a powder.

5. The method of claim 1, wherein said pharmaceutical composition is administered as a gel.

6. The method of claim 1, wherein said pharmaceutical composition comprises carboxymethylated starch, water, and a salt.

7. The method of claim 1, wherein said pharmaceutical composition comprises epichlorohydrin-modified starch, water, and a salt.

8. The method of claim 1, wherein said pharmaceutical composition comprises carboxymethylated starch, epichlorohydrin-modified starch, water, and a salt.

9. The method of claim 1, wherein said starch composition has a molecular weight in the range of 500,000 daltons to 11,000,000 daltons.

10. The method of claim 1, wherein the starch composition has a particle diameter in the range from 30 μm to 100 μm.

11. The method of claim 1, wherein said pharmaceutical composition is particulate carboxymethylated starch and further includes at least one alkali metal salt selected from the group consisting of sodium chloride and potassium chloride, and wherein said carboxymethylated starch has a molecular weight in the range from 500,000 daltons to 11,000,000 daltons, and a particle diameter of 30 microns to 100 microns.

12. The method of claim 1, wherein said carboxymethylated starch is present as sodium salt of a carboxymethyl ether.

13. The method of claim 1, wherein said carboxymethylated starch is made up of about 4.5×10¹⁰ to 2.3×10¹² amylose molecules and 5.6×10⁷ to 1.3×10¹⁰ amylopectin molecules.

14. The method of claim 1, wherein said carboxymethylated starch is 25 to 45 percent crosslinked.

15. The method of claim 1, wherein said carboxymethylated starch includes at least one salt selected from the group consisting of sodium chloride and potassium chloride, in an amount up to 10 weight %, based on the dry weight of the starch.

16. The method of claim 1, wherein said carboxymethylated starch has a pH value in a water solution in the range from 3.0 to 7.5.

17. The method of claim 1, said method comprising the steps:
i) providing a pharmaceutical composition in powder form;
ii) administering the composition of Step i) directly on a anastomosis site in a subject after an anastomosis; and
iii) forming a gel of the composition covering the anastomosis site.

18. The method of claim 1, said method comprising the following steps:
i) providing the pharmaceutical composition in powder form;
ii) premixing the powder of Step i) with an aqueous liquid in an amount sufficient form a gel; and
iii) applying the gel of Step ii) to an anastomosis site.

19. The method of claim 1, said method comprising the following steps:
i) providing the pharmaceutical composition as a gel; and
ii) applying the gel of Step i) to an anastomosis site.

20. The method of claim 17, wherein said gel is formed by application of an aqueous liquid selected from the group consisting of water, and a saline solution.

21. The method of claim 17, wherein said gel is formed by application of an aqueous liquid selected from the group consisting Ringer's solution, Ringer's acetate solution, and Ringer's lactate.

22. The method of claim 18, wherein said aqueous liquid is selected from the group consisting of water and a saline solution which includes one or more cations selected from the group consisting of sodium, potassium, ammonium, magnesium, calcium, iron(II), iron(III), aluminum; and one or more anions selected from the group consisting of fluoride, chloride, bromide, iodide, oxide, sulfide, carbonate, sulfate, phosphate, nitrate, chromate, permanganate, and hexacyanoferrate(II).

23. The method of claim 18, wherein said aqueous liquid is selected from the group consisting Ringer's solution, Ringer's acetate solution, and Ringer's lactate.

24. The method of claim 1, wherein said pharmaceutical composition is in gel form and further comprises one or more cations selected from the group consisting of sodium, potassium, ammonium, magnesium, calcium, iron(II), iron(III), and aluminum; and one or more anions selected from the group consisting of fluoride, chloride, bromide, iodide, oxide, sulfide, carbonate, sulfate, phosphate, nitrate, chromate, permanganate, hexacyanoferrate(II), acetate and lactate.
